SEQUENCE LISTING (10 sequences)

SEQ ID NO: 1 - protein, 10 aa, Homo sapines
GFTFSTYGMH

SEQ ID NO: 2 - protein, 26 aa, Homo sapines
GKGLEWLSYISGGSSYIFYADSVRGR

SEQ ID NO: 3 - protein, 13 aa, Homo sapines
CARILRGGSGMDL

SEQ ID NO: 4 - protein, 15 aa, Homo sapines
CTGSSSNIGAGYNVY

SEQ ID NO: 5 - protein, 7 aa, Homo sapines
GNINRPS

SEQ ID NO: 6 - protein, 12 aa, Homo sapines
CAAWDKSISGLV

SEQ ID NO: 7 - protein, 119 aa, Artificial Sequence
  a heavy chain variable region
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 8 - protein, 112 aa, artificial sequence
  a light chain variable region
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 9 - protein, 449 aa, artificial sequence
  a heavy chain
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHTYQKSLSLSPGK

SEQ ID NO: 10 - protein, 217 aa, Artificial Sequence
  a light chain
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0174658 A1
(43) Pub. Date: Jun. 8, 2023

(54) METHOD OF PROVIDING SAFE ADMINISTRATION OF AN ANTI-CD40 ANTIBODY

(71) Applicant: JANSSEN BIOTECH, INC., Horsham, PA (US)

(72) Inventor: Michael STREIT, Horsham, PA (US)

(73) Assignee: JANSSEN BIOTECH, INC., Horsham, PA (US)

(21) Appl. No.: 17/613,632

(22) PCT Filed: May 27, 2020
(86) PCT No.: PCT/IB2020/055023

§ 371 Date: Nov. 23, 2021

Related U.S. Application Data
(60) Provisional application No. 62/853,488, filed on May 28, 2019.

(51) Int. Cl. C07K 16/28; A61K 39/395; A61K 31/495; A61K 31/47; A61P 35/00; A61K 31/573; A61K 31/138; A61K 31/167; A61K 31/341; A61K 31/4178

(52) U.S. Cl. CPC C07K 16/2878; A61K 39/3955; A61K 31/495; A61K 31/47; A61P 35/00; A61K 31/573; A61K 31/138; A61K 31/167; A61K 31/341; A61K 31/4178; A61K 2039/505

(57) ABSTRACT

Methods for clinically proven safe administration of an anti-CD40 antibody by intravenous administration are provided. Also provided are methods for clinically proven safe treatment of advanced solid tumors by intravenous administration of an anti-CD40 antibody.

### REFERENCE TO SEQUENCE LISTING SUBMITTED ELECTRONICALLY

This application contains a Sequence Listing, which is submitted electronically via EFS-Web as an ASCII formatted sequence listing with a file name “Sequence Listing_688097.0808” creation date of May 22, 2019, and having a size of 10 kb. The sequence listing submitted via EFS-Web is part of the specification and is herein incorporated by reference in its entirety.

### FIELD OF THE INVENTION

CD40, a 48 kilodalton, transmembrane cell-surface glycoprotein is a co-stimulatory receptor belonging to the tumor necrosis factor receptor (TNFR) superfamily (Elgueta R, et al. Immunol Rev., 2009, 229(1):152-172). The constitutive expression of CD40 is diverse and the receptor can be detected on the surface of antigen presenting cells (APC), including dendritic cells (DC), B-lymphocytes, and macrophages. In addition, CD40 is expressed on granulocytes, endothelial cells, smooth muscle cells, fibroblasts, and epithelial cells (Korniluk et al. Tumour Biol., 2014, 35(10):9447-9457; Peters et al. Semin Immunol., 2009, 21(5):293-300).

Consistent with its widespread expression on normal cells, CD40 is also present on the membranes of a wide range of malignant cells, including non-Hodgkin and Hodgkin lymphomas, myeloma, and some carcinomas including nasopharynx, bladder, cervix, kidney, and ovary (Eliopoulos A G & Young L S., Curr. Opin. Pharmacol., 2004, 4(4):360-367). CD40 interacts with a single ligand, CD40L (or CD154), a transmembrane protein that is expressed by activated T-lymphocytes, B-lymphocytes, platelets, mast cells, macrophages, basophils, natural killer (NK) cells, and non-hematopoietic cells (smooth muscle cells, endothelial cells, and epithelial cells). The binding of CD40 to its sole ligand CD40L as part of a cell-cell interaction activates an intracellular signal transduction pathway that involves a series of adapter molecules known as TNF Receptor Activation Factors (or TRAFs). In order to initiate this intracellular signal transduction, multiple CD40 receptors have to form a cluster on the cell membrane (Peters et al. Semin Immunol., 2009, 21(5):293-300). This CD40 clustering allows for a supramolecular signaling complex composed of multiple TRAFs to assemble which in turn leads to the activation of down-stream transcription factors including nuclear factor kappa B (NF-κB) (Kornbluth et al. Int. Rev. Immunol., 2012, 31(4):279-288).

The molecular consequences of CD40 signaling depend on the cell type expressing CD40 and the microenvironment in which the CD40 signal is provided (Vonderheide et al. Clin Cancer Res., 2013, 19(5):1035-1043). CD40 ligation and cross-linking is required for the adaptive immune response through the licensing of APC and especially DC by inducing the up-regulation of membrane co-stimulatory- and MHC-molecules as well as the production of pro-inflammatory cytokines. Thus, CD40 is involved in the functional maturation of APCs and consequently the activation of antigen-specific T-lymphocytes (Long et al. Cancer Discov., 2016, 6(4):400-13; Moran et al. Curr. Opin. Immunol., 2013, 25(2):230-237). CD40 also plays a role in humoral immunity by activating resting B-lymphocytes and by increasing their antigen-presenting function (Vonderheide et al. Clin Cancer Res., 2013, 19(5):1035-1043; Wolchok et al. Clin. Cancer Res., 2009, 15(23):7412-7420). Moreover, CD40 is involved in the induction of innate immunity through a stimulation of cytotoxic myeloid cells such as NK cells, macrophages, and granulocytes (Rakhmilevich et al. Int. Rev. Immunol., 2012, 31(4):267-278). An ambivalent role promoting both tumor progression as well as tumor cell apoptosis has been attributed to the CD40/CD450L pathway in different neoplastic diseases (Korniluk et al. Tumour Biol., 2014, 35(10):9447-9457).

These pivotal CD40/CD40L mediated pathways have ambivalent roles in both, promoting tumor progression as well as inducing tumor cell apoptosis in different neoplastic diseases (Beatty GL, et al. Science, 2011, 331(6024):1612-1616). However, the systemic administration of CD40-antibodies has been associated with adverse side effects, such as shock syndrome, and cytokine release syndrome (van Mierlo et al., 2002, Proc. Natl. Acad. Sci. USA, 99:5561-5566; van Mierlo et al., 2004, J Immunol 173:6753-6759).

In light of the above, there remains a need for improved anti-tumor therapies, particularly anti-CD40 agonist antibodies suitable for clinical use.

### BRIEF SUMMARY OF THE INVENTION

The invention relates to a clinically proven safe administration of an anti-CD40 antibody to subjects, including for clinically proven safe treatment of advanced solid tumors.

In one general aspect, the invention relates to a method of providing clinically proven safe administration of an anti-CD40 antibody to a human subject in need thereof, comprising intravenously administering to the subject a pharmaceutical composition comprising the antibody and a pharmaceutically acceptable carrier, preferably the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 1, 2, and 3, respectively, and the light chain variable region comprising light chain complementarity determining regions (LCDRs) LCDR1, LCDR2 and LCDR3 amino acid sequences of SEQ ID NOs: 4, 5, and 6, respectively, and wherein a total dosage of the antibody administered is 50 μg/kg to 2500 μg/kg, preferably 75 μg/kg to 2000 μg/kg, body weight of the subject per administration.

In one embodiment, the human subject is diagnosed with an advanced solid tumor.

In one embodiment, the anti-CD40 antibody comprises a heavy chain variable region (VH) having the amino acid sequence of SEQ ID NO: 7 and a light chain variable region (VL) having the amino acid sequences of SEQ ID NO: 8.

In one embodiment, the anti-CD40 antibody comprises a heavy chain (HC) having the amino acid sequence of SEQ ID NO: 9 and a light chain (LC) having the amino acid sequences of SEQ ID NO: 10.

In some embodiments, the total dosage of the anti-CD40 antibody administered per administration is 75 μg/kg, 200 μg/kg, 400 μg/kg, 600 μg/kg, 700 μg/kg, 800 μg/kg, 900 μg/kg, 1000 μg/kg, 1100 μg/kg, 1200 μg/kg, 1300 μg/kg, 1400 μg/kg, 1500 μg/kg, 1800 μg/kg, or 2000 μg/kg body weight of the subject, or any dosage in between.

In one embodiment, the total dosage of the pharmaceutical composition is intravenously administered to the human subject over about 2 hours, preferably the pharmaceutical composition is intravenously administered to the human subject repeatedly, more preferably once every two weeks.

In one embodiment, the method further comprises administering to the human subject a therapeutic agent before or after the administration of the anti-CD40 antibody, preferably the therapeutic agent is selected for the group consisting of corticosteroid, antihistamine, antipyretic, H2-antagonist, and antiemetic.

In one embodiment, the pharmaceutical composition comprises 10 mg/ml to 100 mg/ml of the anti-CD40 antibody, such as 10 mg/ml, 20 mg/ml, 30 mg/ml, 40 mg/ml, 50 mg/ml, 60 mg/ml, 70 mg/ml, 80 mg/ml, 90 mg/ml or 100 mg/ml, and a pharmaceutically acceptable carrier.

In another general aspect, the invention relates to a method of providing clinically proven safe administration of an anti-CD40 antibody to a human subject in need thereof, comprising intravenously administering to the subject a pharmaceutical composition comprising the antibody and a pharmaceutically acceptable carrier, preferably the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 1, 2, and 3, respectively, and the light chain variable region comprising light chain complementarity determining regions (LCDRs) LCDR1, LCDR2 and LCDR3 amino acid sequences of SEQ ID NOs: 4, 5, and 6, respectively, wherein a total dosage of the antibody administered is about 600 μg/kg to about 900 μg/kg body weight of the subject per administration, preferably the human subject is diagnosed with non-small cell lung cancer (NSCLC), pancreatic cancer, or cutaneous melanoma.

In one embodiment, the anti-CD40 antibody comprises a heavy chain variable region (VH) having the amino acid sequence of SEQ ID NO: 7 and a light chain variable region (VL) having the amino acid sequences of SEQ ID NO: 8.

In one embodiment, the anti-CD40 antibody comprises a heavy chain (HC) having the amino acid sequence of SEQ ID NO: 9 and a light chain (LC) having the amino acid sequences of SEQ ID NO: 10.

The details of one or more embodiments of the invention are set forth in the description below. Other features and advantages will be apparent from the following detailed description, and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

The patent or application file contains at least one drawing executed in color. Copies of this patent or patent application publication with color drawings will be provided by the Office upon request and payment of the necessary fess.

FIG. 1 shows a diagrammatic representation of the study design of the clinical study in Example 1. IV=intravenous; NSCLC=non-small cell lung cancer; q14d=every 14 days; RP2D=recommended Phase 2 dose.

FIG. 2 shows the study design and cohorts with and without pre-infusion of corticosteroids.

FIG. 3 shows the infusion-related reactions (IRRs) incidence per assigned dose and censored with dose escalation.

FIG. 4 demonstrates mean serum concentration over time cycle 1 and 2.

FIG. 5 demonstrates dose-normalized AUC_{0-24 h}.

FIGS. 6A-C demonstrate proportion of B cells (FIG. 6A), T Cells (FIG. 6B), and NK (FIG. 6C) cells in peripheral blood following infusion with ANTIBODY A normalized to pre-infusion levels (cohorts without corticosteroids).

FIGS. 7A-I demonstrate cytokine/chemokine levels in peripheral blood following infusion with ANTIBODY A (cohorts without corticosteroids): MCP-1 (FIG. 7A), IP-10 (FIG. 7B), MIP-1β (FIG. 7C), IFN-γ (FIG. 7D), MIP-1α (FIG. 7E), IL-8 (FIG. 7F), TNF-α (FIG. 7G), IL-6 (FIG. 7H), and IL12p70 (FIG. 7I).

FIGS. 8A-D demonstrate expression of activation/maturation markers on peripheral blood B lymphocytes: HLA-DR (FIG. 8A), CD54 (FIG. 8B), CD80 (FIG. 8C), and CD86 (FIG. 8D). Symbols and lines represent each individual patient of cohort 6B Expansion (see FIG. 2). Note: Fold change (24-h post-infusion of ANTIBODY A vs pre-infusion) in intensity of staining was calculated for each marker and converted to Loge scale. Of the 6 patients in the final cohort (1200 μg/kg without corticosteroids), 4 patients had useable data and were graphed accordingly.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “subject” refers to a mammalian subject, preferably human, diagnosed with or suspected of having an IFN-I mediated disease, whom will be or has been administered an anti-IFN-α/-ω antibody according to a method of the invention. Diagnosis of an IFN-I mediated disease can be done by a clinician according to clinical diagnostic testing, physical examination of the subject, or any other accepted method for diagnosing a subject with a particular disease.

As used herein, “CD40,” refers to a cell-surface expressed glycoprotein that belongs to the tumor necrosis factor receptor (TNFR) superfamily and plays a central role in the immune system. It is expressed on a variety of immune cells, such as B cells, Dendritic cells, monocytes, and macrophages, and professional APCs, are activated when signaling via CD40 occurs (reviewed by Tasci et al. Cell. Mol. Life. Sci., 2001, (58): 4-43). CD40 expression occurs in many normal cells and tumor cells, such as B-lymphomas, solid tumors, melanomas and carcinomas. It is well-established that activation of CD40 is effective in triggering anti-tumor responses, and CD40 activation contributes to tumor growth impairment by at least the mechanisms of immune activation, a direct apoptotic effect on CD40-positive tumors and stimulation of a humoral response leading to antibody-dependent cell-mediated cytotoxicity (ADCC) and complement dependent cytotoxicity (CDC).

“CD40” as used herein includes any natural or synthetic protein with structural and/or functional identity to the human CD40 protein as defined herein and/or natural variants thereof. Preferably, the CD40 is human CD40, such as UniProt Accession No. P25942 and GenBank Accession No. AAH12419.

As used herein, an “an anti-CD40 antibody,” refers to an agonistic, human monoclonal antibody (mAb) of the IgG1 subtype, or antigen binding fragment thereof, that binds and enhance the effects of the natural ligand CD40L. The agonistic CD40 antibody of this invention may induce direct anti-tumor effects (1) through binding to CD40 receptors expressed on tumor cells and indirect anti-tumor effects, (2) through the ‘licensing’ of DC and the activation of cytotoxic T-cells (CTL), as well as (3) through the activation of cytotoxic myeloid cells such as NK cells or tumor macrophages. In a preferred embodiment, the anti-CD40 antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 1, 2, and 3, respectively, and the light chain variable region comprising light chain complementarity determining regions (LCDRs) LCDR1, LCDR2 and LCDR3 amino acid sequences of SEQ ID NOs: 4, 5, and 6, respectively. In another preferred embodiment, the anti-CD40 antibody comprises a heavy chain variable region (VH) having the amino acid sequence of SEQ ID NO: 7 and a light chain variable region (VL) having the amino acid sequences of SEQ ID NO: 8. In another preferred embodiment, the anti-CD40 antibody comprises a heavy chain (HC) having the amino acid sequence of SEQ ID NO: 9 and a light chain (LC) having the amino acid sequences of SEQ ID NO: 10.

Additional anti-CD40 antibodies or antigen binding fragments thereof that can be used in the present invention include those described in U.S. Pat. No. 9,676,862, which is herein incorporated by reference.

Anti-CD40 antibodies can be prepared by any method known in the art in view of the present disclosure for preparing monoclonal antibodies including, but not limited to, hybridoma production. For example, anti-CD40 antibodies can be produced in a mammalian cell line (e.g., Chinese Hamster Ovary (CHO) cell line) using recombinant DNA technology. In particular, methods of producing anti-CD40 antibodies useful for the invention are further described in, e.g., U.S. Pat. No. 9,676,862, which is herein incorporated by reference.

The term “safe,” as it relates to a dose, dosage regimen, treatment or method with an anti-CD40 antibody refers to a favorable risk:benefit ratio with an acceptable frequency and/or acceptable severity of treatment-emergent adverse events (referred to as AEs or TEAEs) compared to the standard of care or to another comparator in accordance with the Federal Food, Drug, and Cosmetic Act, as amended (secs. 201-902, 52 Stat. 1040 et seq., as amended; 21 U.S.C. §§ 321-392). In particular, safe as it relates to a dose, dosage regimen, or treatment with an anti-CD40 antibody of the present invention refers to with an acceptable frequency and/or acceptable severity of adverse events associated with administration of the antibody if attribution is considered to be possible, probable, or very likely due to the use of the anti-CD40 antibody. Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug.

As used herein, unless otherwise noted, the term “clinically proven” (used independently or to modify the term “safe”) shall mean that it has been proven by a clinical trial wherein the clinical trial has met the approval standards of U.S. Food and Drug Administration, European Medicines Evaluation Agency (EMEA) or a corresponding national regulatory agency. In the instant invention, the clinical study is a phase 1, open-label study of the safety, pharmacokinetics, and pharmacodynamics of ANTIBODY A, an agonistic human monoclonal antibody targeting CD40 in patients with advanced stage solid tumors.

As used herein, the phrases “adverse event,” “treatment-emergent adverse event,” and “adverse reaction” mean any harm, unfavorable, unintended or undesired sign or outcome associated with or caused by administration of a pharmaceutical composition or therapeutic. However, abnormal values or observations are not reported as adverse events unless considered clinically significant by the investigator. As used herein, when referring to an adverse event, “clinically apparent” means clinically significant as determined by a medical doctor or an investigator using standard acceptable to those of ordinary skill in the art. When the harm or undesired outcome of adverse events reaches such a level of severity, a regulatory agency may deem the pharmaceutical composition or therapeutic unacceptable for the proposed use. Examples of adverse events or reactions when used in the context of intravenous administration of an anti-CD40 antibody include, but are not limited to, infections and infestations, such as rhinitis, herpes zoster, and myringitis bullosa; respiratory, thoracic and mediastinal disorders, such as cough, throat irritation, and oropharyngeal pain; gastrointestinal disorders, such as diarrhea and flatulence; nervous system disorders, such as headache and dizziness; blood and lymphatic system disorders, such as anaemia and lymphadenopathy; back pain, premature labour, infusion reactions, local injection site reactivity, malignancy and no anaphylactic or serum sickness-type reactions.

As used herein, “treatment” or “treat” refers to therapeutic treatment. Individuals in need of treatment include those subjects diagnosed with the disorder or a symptom of the disorder. Subjects that may be treated also include those prone to or susceptible to have the disorder, of those in which the disorder is to be prevented. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. Beneficial clinical result includes, in a subject who has received treatment, for example reduced proliferation of B cells or dendritic cells, reduction of inflammatory cytokines, adhesion molecules, proteases, immunoglobulins, combinations thereof, increased production of anti-inflammatory proteins, a reduction in the number of autoreactive cells, an increase in immune tolerance, inhibition of autoreactive cell survival, and/or a decrease in one or more symptoms mediated by CD40/CD40L mediated pathways. Clinical response may be assessed using screening techniques such as magnetic resonance imaging (MM) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

The terms “efficacy” and “effective” as used herein in the context of a dose, dosage regimen, treatment or method refer to the effectiveness of a particular dose, dosage or treatment regimen. Efficacy can be measured based on change in the course of the disease in response to an agent of the present invention. For example, an anti-CD40 antibody of the present invention (e.g., ANTIBODY A) is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, an anti-CD40 antibody of the present invention can be administered to achieve an improvement in a subject's condition related to advanced solid tumors.

Disease evaluations for advanced solid tumors include computed tomography (CT) or magnetic resonance imaging (MM) for all subjects and bone scans for subjects with prostate cancer. Disease response are evaluated according to Response Evaluation Criteria In Solid Tumors (RECIST) v1.1 criteria and according to Immune-Related Response Criteria (irRC). Prostate Cancer Clinical Trials Working Group 3 (PCWG3) criteria35 are used to evaluate disease response for subjects with prostate cancer.

As used herein, a dosage amount of an anti-CD40 antibody in “m/kg” refers to the amount of the anti-CD40 antibody in micrograms per kilogram of the body weight of a subject to be administered with the antibody.

In one general aspect, the invention relates to a method of providing clinically proven safe intravenous administration of an anti-CD40 antibody to a subject, preferably a human subject, in need thereof. Preferably, the subject is diagnosed with any type of advanced or refractory solid tumor malignancy that is metastatic or unresectable. Examples of the above disease include, but are not limited to, bladder cancer, breast cancer, cervical cancer, colon & rectal, endometrial cancer, kidney cancer, lip & oral cancer, liver cancer, melanoma, mesothelioma, non-small cell lung cancer, nonmelanoma skin cancer, oral cancer, ovarian cancer, pancreatic cancer, prostate cancer, sarcoma, small cell lung cancer, and thyroid cancer.

In one embodiment, a method of providing clinically proven safe administration of an anti-CD40 antibody to a subject and/or safe treatment of an advanced solid tumor in a subject, preferably a human subject, comprises intravenously administering to the subject a pharmaceutical composition comprising an anti-CD40 antibody and a pharmaceutically acceptable carrier, wherein a total dosage of the anti-CD40 antibody administered is 50 μg/kg to 2500 μg/kg, preferably 75 μg/kg to 2000 μg/kg mg/kg, body weight of the subject per administration.

Intravenous administration refers to administration directly into a vein. Intravenous administration can be via injection (e.g., with a syringe at higher pressures) or via infusion (e.g., using the pressure supplied by gravity). Intravenous administration is typically the quickest method for delivering a drug or therapeutic throughout the body, because the drug or therapeutic is carried by circulation. When administration of an anti-CD40 antibody is via intravenous administration, administration can be by intravenous infusion or injection, and is preferably via infusion. For example, the total dosage of an anti-CD40 antibody to be administered to the subject per administration can be administered by intravenous infusion over a period of about 30 minutes to 180 minutes, preferably 60 minutes to 120 minutes, such as 30 minutes, 60 minutes, 90 minutes, 120 minutes, 150 minutes, or 180 minutes.

The total dosage of an anti-CD40 antibody per administration is selected so as to provide safe administration and/or safe treatment by intravenous administration as determined in clinical trials. According to embodiments of the invention, when the pharmaceutical composition is administered intravenously, a total dosage of the anti-CD40 antibody administered per administration is, for example, 50 μg/kg, 75 μg/kg, 200 μg/kg, 400 μg/kg, 600 μg/kg, 700 μg/kg, 800 μg/kg, 900 μg/kg, 1000 μg/kg, 1100 μg/kg, 1200 μg/kg, 1300 μg/kg, 1400 μg/kg, 1500 μg/kg, 1800 μg/kg, or 2000 μg/kg, or any dosage in between.

The total dosage of the anti-CD40 antibody can be administered once per day, once per week, once per two weeks, once per month, once every six months, etc. for a period of one day, one week, one month, six months, 1 year, 2 years or longer. For example, a total dosage of 75 μg/kg to 2000 μg/kg of the anti-CD40 antibody can be administered per administration (e.g., once per day for at least one day) by a single intravenous injection, i.e., over a time of period of 0 minutes to 3 hours, such as 5 minutes, 10 minutes, 15 minutes, 20 minutes, 25 minutes, 30 minutes, 35 minutes, 40 minutes, 45 minutes, 50 minutes, 55 minutes, 1 hour, 2 hours, or 3 hours. Multiple administrations of the anti-CD40 antibody, each at a total dosage of 75 μg/kg to 2000 μg/kg, can be administered to a subject in need thereof.

Pharmaceutical compositions suitable for use in the methods of the invention are formulated for intravenous administration. Examples of formulations suitable for intravenous administration include, but are not limited to, solutions, suspensions, emulsions, and dry products that can be dissolved or suspended in a pharmaceutically acceptable carrier for injection or infusion. In a preferred embodiment, a pharmaceutical composition comprising an anti-CD40 antibody for use in the methods of the invention is formulated as a solution.

A concentration of an anti-CD40 antibody included in pharmaceutical compositions used in the invention can vary. Typically, the concentration of the anti-CD40 antibody is 1 mg/mL to 100 mg/mL, such as 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL, or any concentration in between. In one embodiment, the concentration of the anti-CD40 antibody is 10 mg/mL to 30 mg/mL, for instance 20 mg/mL. In one embodiment, the concentration of the anti-CD40 antibody is 20 mg/mL to 60 mg/mL, for instance 40 mg/mL

Pharmaceutical compositions for use in the invention further comprise one or more pharmaceutically acceptable carriers, such as those widely employed in the art of drug manufacturing, and particularly antibody drug manufacturing. As used herein, the term “carrier” refers to any excipient, diluent, buffer, stabilizer, or other material well known in the art for pharmaceutical formulations. Pharmaceutically acceptable carriers in particular are non-toxic and should not interfere with the efficacy of the active ingredient. The pharmaceutically acceptable carriers include excipients and/or additives suitable for use in the pharmaceutical compositions known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference.

According to embodiments of the invention, a pharmaceutical composition for use in the invention comprises an anti-CD40 antibody and a pharmaceutically acceptable carrier. In some embodiments, the pharmaceutically acceptable carrier comprises one or more amino acids, such as L-histidine and glycine, one or more carbohydrates, such as lactose, maltose, sucrose, and trehalose, one or more surfactants, such as polysorbate 20 and polysorbate 80, and one or more alcohol, such as D-sorbitol. Preferably, the pharmaceutical composition has a pH of 5 to 6, such as a pH of 5.0, 5.1, 5.2, 5.3, 5.4, 5.5, 5.6, 5.7, 5.8, 5.9, or 6.0, or any value in between.

In some embodiments, a pharmaceutical composition for use in the invention comprises one or more amino acids, such as L-histidine and glycine. The amino acid can be present at a concentration of 1 mM to 40 mM, 1mM to 20 mM, or 20 mM to 40 mM, or 0.50% to 2.00% weight by volume (w/v). For example, the pharmaceutical composition can comprise L-histidine at a concentration of 1 mM, 5 mM, 10 mM, 15 mM, 20 mM, 25 mM, 30 mM, 35 mM, or 40 mM, or any concentration in between. In another example, the pharmaceutical composition can comprise glycine at a concentration of 0.50% (w/v), 0.75% (w/v), 1.00% (w/v), 1.25% (w/v), 1.50% (w/v), 1.75% (w/v), or 2.00% (w/v), or any concentration in between.

In some embodiments, a pharmaceutical composition for use in the invention comprises a sugar, such as sucrose, glucose, cellobiose, or trehalose, at a concentration of 1% to 10% weight by volume (w/v), 5% to 10% (w/v), or 8% to 9% (w/v). For example, the pharmaceutical composition can comprise sucrose, cellobiose and/or trehalose at a concentration of 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v), or any concentration in between.

In some embodiments, a pharmaceutical composition for use in the invention comprises a surfactant, such as polysorbate 80 (PS80) or polysorbate 20 (PS20), at a concentration of 0.01% (w/v) to 0.10% (w/v), 0.01% (w/v) to 0.08% (w/v), or 0.02% (w/v) to 0.05% (w/v). For example, the concentration of polysorbate 20 and/or polysorbate 80 can be 0.01%, 0.02%, 0.03%, 0.04%, 0.05%, 0.06%, 0.07%, 0.08%, 0.09% or 0.1% (w/v), or any concentration in between.

In some embodiments, a pharmaceutical composition for use in the invention comprises a polyol, such as mannitol, xylitol or D-sorbitol at a concentration of 0.01% (w/v) to 0.10% (w/v), 0.01% (w/v) to 0.08% (w/v), or 0.02% (w/v) to 0.05% (w/v). For example, the concentration of the mannitol, xylitol or D-sorbitol can be 0.01%, 0.02%, 0.03%, 0.04%, 0.05%, 0.06%, 0.07%, 0.08%, 0.09% or 0.1% (w/v), or any concentration in between.

Pharmaceutical compositions comprising an anti-CD40 antibody for use in the invention can be prepared by any method known in the art in view of the present disclosure. For example, an anti-CD40 antibody can be mixed with one or more pharmaceutically acceptable carriers to obtain a solution. The solution can be stored as a frozen liquid at a controlled temperature ranging from −40° C.±10° C. to −70° C.±20 ° C. and under protection from light exposure in an appropriate vial until administered to the subject.

According to embodiments of the invention, pre-infusion and post-infusion supportive therapy can be used for the treatment of advanced solid tumors in addition to the administration of the anti-CD40 antibody. In some embodiments, the human subject receives pre-infusion medications prior to the administration of anti-CD40 antibody. In some embodiments, the human subject receives post-infusion medications after the administration of anti-CD40 antibody. Examples of these medications include, but are not limited to, corticosteroid, antihistamine, antipyretic, H₂-antagonist, and antiemetic.

According to embodiments of the invention, a variety of factors can be analyzed to determine by clinical trials such as those described herein whether a particular dosage of the anti-CD40 antibody provides for safe intravenous administration. For example, safety of a certain dosage of intravenously administered anti-CD40 antibody can be assessed by immunogenicity studies (e.g., measuring the production of antibodies to the anti-CD40 antibody); by determining the dose limiting toxicities (DLT) in the subject; by determining the effects on blood biomarkers, such as serum proteins (e.g., cytokines, chemokines, and inflammatory proteins) by protein profiling; by pharmacokinetic studies (e.g., an area under the concentration time curve (AUC), and a maximum concentration observed (Cₘₐₓ). The safety of intravenously administered anti-CD40 antibody can also be monitored by physical examination of the subject; observation of local injection site reactions, systemic injection related reactions, and other allergic reactions; electrocardiograms; clinical laboratory tests; vital signs; and monitoring of other adverse events, such as infusion related reactions (IRRS).

In some embodiments, clinically proven safe administration of an anti-CD40 antibody and/or clinically proven safe treatment of an advanced solid tumor is determined by measuring amounts of antibodies to anti-CD40 antibody in a sample obtained from a subject. The amounts of antibodies to anti-CD40 antibody can be measured by any method known in the art in view of the present disclosure, e.g., ELISA.

In some embodiments, clinically proven safe administration of an anti-CD40 antibody and/or clinically proven safe treatment of an advanced solid tumor is determined by assessing pharmacokinetic parameters such as terminal half-life, target saturation, an area under the concentration time curve (AUC), and a maximum concentration observed (Cₘₐₓ). Serum samples are analyzed to determine concentrations of the anti-CD40 antibody by any method known in the art in view of the present disclosure. The pharmacokinetic parameters are then analyzed, for example by non-compartment analysis (NCA), to calculate pharmacokinetic parameters, such as AUC, Cₘₐₓ, terminal half-life (T_{1/2}), total systemic clearance after intravenous administration (CL), volume of distribution at terminal phase (V_{z}), total systemic clearance over bioavailability (CL/F), and volume of distribution at terminal phase over bioavailability (V_{z}/F).

In some embodiments, clinically proven safe administration of an anti-CD40 antibody and/or clinically proven safe treatment of an advanced solid tumor exhibits target-mediated drug disposition with rapid decline in serum concentration. For example, the half-life of the anti-CD40 antibody is about 10-16 hours when the total dosage of the anti-CD40 antibody administered per administration is about 600 μg/kg body weight of the subject, preferably about 13 hours, or the anti-CD40 antibody about 20-28 hours when the total dosage of the anti-CD40 antibody administered per administration is no less than 1200 μg/kg body weight of the subject, preferably 24 hours.

In some embodiments, clinically proven safe administration of an anti-CD40 antibody and/or clinically proven safe treatment of an advanced solid tumor achieves target saturation at a concentration of 1000 μg/kg to 1400 μg/kg body weight of the subject. For example, the saturation concentration can be 1000 μg/kg, 1050 μg/kg, 1100 μg/kg, 1150 μg/kg, 1200 μg/kg, 1250 μg/kg, 1300 μg/kg, 1350 μg/kg, or 1400 μg/kg, or any dosage in between.

According to the embodiments of this invention, increases in mean Cₘₐₓ and AUC_{0-24 h} are more than dose-proportional at doses <1200 μg/kg and dose proportional at doses ≥1200 μg/kg. In some embodiments, clinically proven safe administration of an anti-CD40 antibody and/or clinically proven safe treatment of an advanced solid tumor is determined by assessing CD40 receptor occupancy. For example, the proportions of B cells, T Cells, and NK cells in peripheral blood following infusion of the anti-CD40 antibody are measured as normalized to pre-infusion levels. According to the embodiments of the invention, a dose-independent margination of B cells, T cells, and natural killer (NK) cells is achieved following the infusion of anti-CD40 antibody, while the dose-dependent B cell recovery is achieved, consistent with observations for competitor anti-CD40 agonist antibodies. NK cells and T cells have decreased in number in the peripheral blood following infusion, and the tested T cells and natural killer (NK) cells are subsequently fully recovered.

In some embodiments, clinically proven safe administration of an anti-CD40 antibody and/or clinically proven safe treatment of an advanced solid tumor is assessed by measuring a panel of serum cytokines and chemokines including, but not limited to MCP-1, IP-10, MIP-1β, MIP-1α, IFN-γ, TNF-α, IL12p70, IL-2, IL-6, IL-8, and IL-12. These data complement flow cytometry studies of B cells, T cells, myeloid, and NK compartments to demonstrate PD changes in cell counts and/or activation status following the infusion of anti-CD40 antibody. According to the embodiments of the invention, after the infusion of the anti-CD40 antibody, peripheral chemokines (MCP-1, IP-10, and MIP-1β) are prominent in the peripheral blood, peaking 1-4 h post-infusion, which is consistent with myeloid cell activation; cytokines (IFN-γ, TNF-α and IL12p70) and chemokines (MIP-1α and IL-8) are also observed, but to a lesser extent; levels of IL-6, which has been associated with the induction of cytokine storm with other CD40 agonist antibodies, is not abundant following the infusion.

In some embodiments, clinically proven safe administration of an anti-CD40 antibody and/or clinically proven safe treatment of an advanced solid tumor is assessed by measuring the licensing of APC/DC and activation of assorted B and T cell subsets in blood samples using appropriate methodology such as, but not restricted to, flow cytometry of cell surface activation markers such as HLA-DR, CD54, CD80 and CD86. According to the embodiments of the invention, these activation markers increase on activated B cells and monocytes following the infusion of the anti-CD40 antibody of the anti-CD40 antibody.

### ABBREVIATIONS

β-hCG β-human chorionic gonadotropin
ADA anti-drug antibodies
ADCC antibody-dependent cell-mediated cytotoxicity
anti-HCV anti-hepatitis-C antibody
APC antigen-presenting cells
AUC area under the serum concentration versus time curve
BLRM Bayesian Logistic Regression Model
CI confidence interval
Cₘₐₓ maximum observed serum concentration
CR complete response
CRF case report form(s) (paper or electronic as appropriate for this study)
CRS cytokine release syndrome
CT computed tomography
CTCAE Common Terminology Criteria for Adverse Events
DC dendritic cells
DLT dose-limiting toxicity
DOR duration of response
ECG Electrocardiogram
ECOG Eastern Cooperative Oncology Group
eDC electronic data capture
EWOC Escalation with Overdose Control
FcγR Fcγ-receptors
FSH follicle stimulating hormone
GCP Good Clinical Practice
GLP Good Laboratory Practice
HBsAg hepatitis B surface antigen
hCD40tg human CD40-transgenic
HIV human immunodeficiency virus
ICF informed consent form
ICH International Conference on Harmonisation
IEC Independent Ethics Committee
IRB Institutional Review Board
irCR immune-related complete response
irRC Immune-Related Response Criteria
IT intratumoral
IV Intravenous
LLOQ lower limit of quantitation
MAD maximum-administered dose
mCRM modified Continual Reassessment Method
MRI magnetic resonance imaging
MTD maximum tolerated dose
NCI National Cancer Institute
NK natural killer
NSCLC non-small cell lung cancer
ORR objective response rate
PCWG Prostate Cancer Clinical Trials Working Group
PD pharmacodynamic(s)
PFS progression-free survival
PK pharmacokinetic(s)
POM proof-of-mechanism
PQC Product Quality Complaint
PR partial response
RBC red blood cell
RECIST Response Evaluation Criteria In Solid Tumors
RP2D recommended Phase 2 dose
SC subcutaneous
SET Safety Evaluation Team
SUSAR suspected unexpected serious adverse reaction
Tₘₐₓ time of maximum observed serum concentration
TRAFs tumor necrosis factor receptor activation factors
Vd volume of distribution
WBC white blood cell

### EXAMPLES

### Example 1: A Phase 1, Open-Label Study of the Safety, Pharmacokinetics, and Pharmacodynamics of ANTIBODY A in Patients with Advanced Stage Solid Tumors

ANTIBODY A is an agonistic, human monoclonal (IgG1) antibody targeting CD40, which is investigated for the treatment of advanced stage solid tumors. This phase 1, open-label study is designed to evaluate the safety, pharmacokinetics, and pharmacodynamics of ANTIBODY A administered as IV infusion in patients with advanced stage solid tumors and to establish the recommended Phase 2 dose (RP2D) and schedule. In Part 2 of the study, additional safety data will be generated and the therapeutic efficacy of ANTIBODY A will be explored in expansion cohorts of subjects with non-small cell lung cancer (NSCLC), pancreatic cancer, and cutaneous melanoma, who have failed or are no longer eligible for approved and effective therapies.

Overview of Study Design

Part 1, Dose Escalation: Escalating ANTIBODY A doses starting from 75 μg/kg will be explored in a modified continual reassessment method (mCRM) design in subjects with advanced stage solid tumors, in order to determine the recommended Phase 2 dose (RP2D). The dose will be increased by not more than half-logarithmical (3.2-fold) dose increments. Dose escalation will continue until the maximum-tolerated dose (MTD) and/or RP2D of ANTIBODY A are defined or the maximum-administered dose (MAD) has been reached.
MTD is the highest ANTIBODY A dose that emerges from the evaluation of PK/PD and safety data guided by the statistical model (BLRM) with EWOC principle during the DLT-evaluation period.
MAD is defined as the highest ANTIBODY A dose administered.
The RP2D will be determined after review of all available PK/PD, safety, and efficacy data in accordance with the Bayesian Logistic Regression Model (BLRM).

Part 2, Dose Expansion: ANTIBODY A will be administered at the RP2D in expansion cohorts of approximately 30 subjects each, in order to further characterize the safety and PK/pharmacodynamic (PD) characteristics and to assess efficacy of this agent in subjects with NSCLC, pancreatic cancer, and cutaneous melanoma.

Part 2 (Dose Expansion) will start after RP2D determination to (1) collect additional information on the safety and PK/PD characteristics of ANTIBODY A in the selected disease populations and (2) to evaluate the clinical activity of the study drug in subjects with NSCLC, pancreatic cancer, and cutaneous melanoma. The expansion cohorts will consist of approximately 30 subjects each.

Biomarker Substudy: additional biomarkers will be assessed to define the impact of ANTIBODY A on innate and adaptive immune responses in tumors.

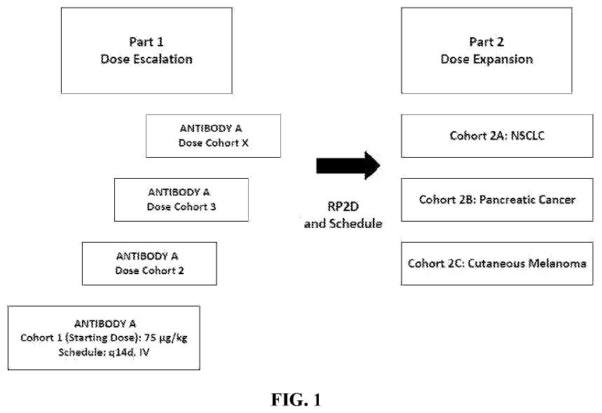

A diagram of the study design is provided in FIG. 1.

Subjects

Subjects must be age 18 years or older and have Eastern Cooperative Oncology Group (ECOG) performance status of 0 or 1.

Part 1: Subjects with any type of advanced or refractory solid tumor malignancy that is metastatic or unresectable are eligible for enrollment in Part 1. The subjects have received all standard treatment options or are no longer eligible for additional standard treatment options.

Part 2: Subjects with histologically or cytologically confirmed NSCLC, pancreatic cancer, or cutaneous melanoma are eligible for enrollment in Part 2. The subject cohorts include, e.g.,

Cohort 2A:
Histologically or cytologically confirmed NSCLC
Stage IV disease
Received at least 2 prior lines of approved, systemic therapy, of which 1 therapy has to be a platinum-containing regimen
At least 1 measurable tumor lesion per RECIST v1.1

Cohort 2B:
Histologically or cytologically confirmed adenocarcinoma of the pancreas
Unresectable, locally advanced (Stage III), or metastatic (Stage IV) disease
Received at least 1 prior line of approved, systemic therapy
At least 1 measurable tumor lesion per RECIST v1.1

Cohort 2C:
Histologically or cytologically confirmed cutaneous melanoma
Unresectable (Stage III) or metastatic (Stage IV) disease
Received at least 1 prior line of approved, systemic therapy
At least 1 measurable tumor lesion per RECIST v1.1

The study of part 2 is ongoing.

Study Agent

ANTIBODY A is supplied as a lyophilized cake or as a frozen liquid. The formulation contains 20 mg/mL or 40 mg/mL ANTIBODY A.

### Dosages and Administration

Doses will be escalated from a starting dose of 75 μg/kg. Administration is by IV infusion. The initial schedule of administration is every 14 days (Q14d) (Days 1 and 15) of the 28-day cycle. Administration is by IV infusion initially over 2 hours.

Recommended Pre- and Post-infusion Supportive Therapy

Prior to each infusion of ANTIBODY A, subjects will receive pre-infusion medications as noted in Table 1.

TABLE 1
Pre-infusion Medications

| Medication | Dose | Administration |
|---|---|---|
| Corticosteroid | dexamethasone | IV - start infusion at least |
| | (20 mg) or | 1 hour prior to study drug |
| | methylprednisolone | |
| | (80 mg) | |
| Antihistamine | diphenhydramine | Oral - administer at least |
| | (50 mg) | 1 hour prior to study drug |
| | or equivalent | IV - start infusion at least |
| | | 30 minutes prior to study drug |
| Antipyretic | Acetaminophen | Oral acetaminophen or IV |
| | (650 mg to | paracetamol - administer at |
| | 1,000 mg) or | least 30 minutes prior to |
| | equivalent | study drug |
| H₂-antagonist^{a} | ranitidine or | IV - start infusion approximately |
| | equivalent | 30 minutes prior to study drug |
| | (50 mg) | |
| Antiemetic^{a} | ondansetron | IV - start infusion approximately |
| | (16-24 mg) or | 30 minutes prior to study drug |
| | equivalent | |

IV = intravenous
^{a}Optional if not explicitly mandated by the Safety Evaluation Team (SET)

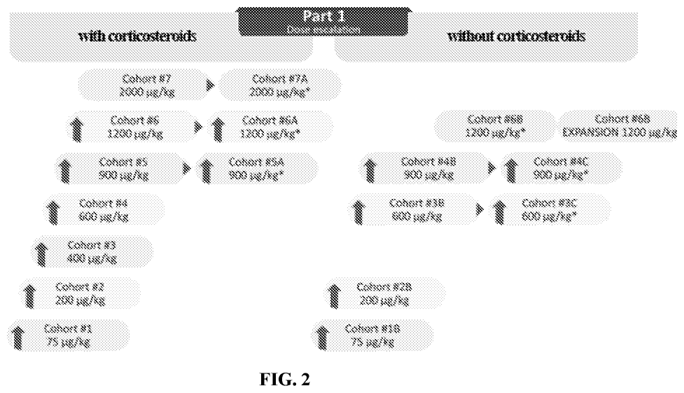

The study design and cohorts with and without pre-infusion of corticosteroids is demonstrated in FIG. 2

Following each infusion of ANTIBODY A, subjects who have experienced ANTIBODY A-related toxicities during or following a prior administration should receive post-infusion medications as noted in Table 2.

TABLE 2
Post-infusion Medications^{a}

| Medication | Dose | Administration |
|---|---|---|
| Corticosteroid ^{b} | dexamethasone (4 mg) twice | as clinically indicated |
| | daily ^{c} | |
| Antihistamine | diphenhydramine (50 mg) or | as clinically indicated |
| | equivalent | |
| Antipyretic | Acetaminophen (650 mg to | as clinically indicated |
| | 1,000 mg) or equivalent | |
| Antipyretic | ondansetron (8 mg) or | as clinically indicated |
| | equivalent (long or | |
| | short acting agents) | |

IV = intravenous
^{a}In the absence of symptoms of JNJ-64457107-related toxicity, post-infusion medication may by given for up to 48 hours after the end of the infusion
^{b} The investigator should use clinical judgment if further corticosteroid support is necessary.
^{c} Oral or IV starting 12 hours after administration of the first dose of study drug. Recommend to limit to 48 hours after JNJ-64457107 adminstration.

### Evaluations

Safety Evaluations

Safety assessments will be based on medical review of adverse event reports and the results of clinical laboratory tests, electrocardiograms (ECGs), vital sign measurements, physical examinations, ECOG performance status, and other safety evaluations at time points.

Pharmacokinetic and Immunogenicity

For all subjects participating in the study, blood or serum samples were used to evaluate the PK, as well as the immunogenicity of ANTIBODY A. Venous blood samples will be collected for measurement of serum concentrations of ANTIBODY A (approximately 5 mL) and for evaluation of anti-ANTIBODY A antibodies (approximately 7.5 mL for combined PK and immunogenicity samples otherwise 5 mL for immunogenicity sample alone). Venous blood samples will be collected and each serum sample will be divided into 3 equal aliquots (1 each for PK, anti-ANTIBODY A antibodies, and a back-up) for time points when both PK and immunogenicity samples are collected, otherwise 2 equal aliquots when only PK samples are collected.

Serum samples will be analyzed to determine concentrations of ANTIBODY A using a validated, specific, and sensitive assay method by or under the supervision of the sponsor. The detection and characterization of antibodies to ANTIBODY A will be performed using a MescoScale Discovery (MSD) Platform validated assay method by or under the supervision of the sponsor. All samples collected for detection of antibodies to ANTIBODY A will also be evaluated for ANTIBODY A serum concentration to enable interpretation of the antibody data.

Biomarkers

Biomarkers will be evaluated to investigate the molecular mode(s) of action of ANTIBODY A, as well as to explore biomarkers that could be predictive of response to therapy. The biomarker aims for this study encompass studies of CD40 receptor occupancy, innate immune response, CD40 activation markers, Fc-dependent effector function, and CD40 expression on tumor tissue. An optional biomarker substudy will use pre- and post-drug biopsies to evaluate changes in immune cells following CD40 engagement, with the goal of identifying drug combination opportunities incorporating ANTIBODY A.

Efficiency Evaluations

Efficiency evaluations will include computed tomography (CT) or magnetic resonance imaging (MM) for all subjects and bone scans for subjects with prostate cancer. Disease response will be evaluated according to Response Evaluation Criteria In Solid Tumors (RECIST) v1.1 criteria and according to Immune-Related Response Criteria (irRC). Prostate Cancer Clinical Trials Working Group 3 (PCWG3) criteria35 will be used to evaluate disease response for subjects with prostate cancer. The relationship between PK and PD, including receptor occupancy, will be explored and reported in a separate report.

### Statistical Methods

Data are summarized using descriptive statistics. Continuous variables will be summarized using the number of observations, mean, standard deviation, median, and range as appropriate. Categorical values will be summarized using the number of observations and percentages as appropriate. The distribution of time-to-event endpoints is estimated using the Kaplan-Meier method.

### Results

Demographics & Disposition: 95 patients of age 18-80 years (median 59) were enrolled in 7 cohorts (n=50, 75m/kg — 2000 μg/kg) with corticosteroids and 5 cohorts (n=45, 75 μg/kg-1200 μg/kg) without steroids and received 1-26 (median 3) cycles of ANTIBODY A (FIG. 2). Most patients have been discontinued due to progressive disease (n=62, 76%).

### Safety Results

Majority of adverse events (AEs) were grade 1 (G1) or 2 (G2) and there were limited number of patients with ≥grade 3 (G3) AEs, as shown in Table 3.

TABLE 3
Safety Summary (all treated analysis set)

w & w/o corticosteroids N = 95

| | All grades | Grade 3 or |
|---|---|---|
| TEAEs, n(%) | (≥10%) | higher (≥5%) |
| Patients with 1 | 94 (98.9) | 53 (55.8) |
| or more TEAEs | | |
| Fatigue | 41 (43.2) | 3 (3.2) |
| Pyrexia | 39 (41.1) | 2 (2.1) |
| Pruritus* | 37 (38.9) | 1 (1.1) |
| Chills | 26 (27.4) | 1 (1.1) |
| Headache | 25 (26.3) | 3 (3.2) |
| Nausea | 21 (22.1) | 0 |
| Rash** | 20 (21.1) | 0 |
| ALT increased | 18 (18.9) | 2 (2.1) |
| AST increased | 16 (16.8) | 6 (6.3) |
| Vomiting | 15 (15.8) | 2 (2.1) |
| Flushing | 14 (14.7) | 0 |
| Decreased appetite | 13 (13.7) | 0 |
| Back pain | 12 (12.6) | 2 (2.1) |
| GGT increased | 12 (12.6) | 7 (7.4) |
| Abdominal pain | 11 (11.6) | 1 (1.1) |
| Diarrhea | 11 (11.6) | 0 |
| Erythema† | 11 (11.6) | 0 |
| Anemia | 8 (8.4) | 5 (5.3) |

*Pruritus includes Pruritus and Prurtitus Generalized;
**Rash includes Rash, Rash Generalized and Rash Maculo-Papular;
†Erythema includes Generalized Erythema and Erythema; Adverse events are coded using MedDRA Version 21.1;
Note:
Patients are counted only once for any given event, regardless of the number of times they actually experienced the event.
AE, adverse event;
ALT, Alanine aminotransferase;
AST, Aspartate aminotransferase
GGT, Gamma-glutamyltransferase;
TEAE, treatment-emergent adverse event

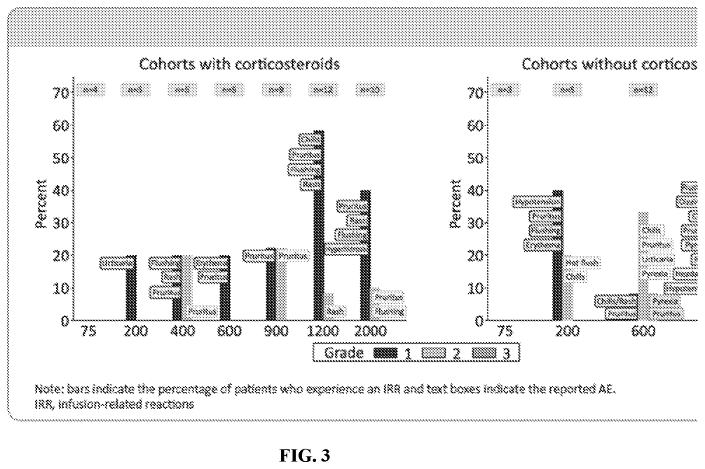

As shown in FIG. 3, Infusion related reactions (IRRs) were reported in 51% of patients (G1-2: 50%; G3: 1%). Most common IRRs (>10%) were pruritus (31%), rash (15%), chills (13%) and flushing (12%). Based on the high pruritus incidence, a new pre-medication plan was implemented. Cetirizine and Montelukast were administered as pre/post-medication on days −3 up to +3 of each ANTIBODY A infusion, and was demonstrated to significantly reduce IRRs and no pruritus has been reported.

Two dose limiting toxicities (DLT) were reported: G3 headache lasting 5 days at 1200 μg/kg with corticosteroids; and G3 ALT/AST+G2 bilirubin increase at 1200 μg/kg without corticosteroids.

Pharmacokinetics Results

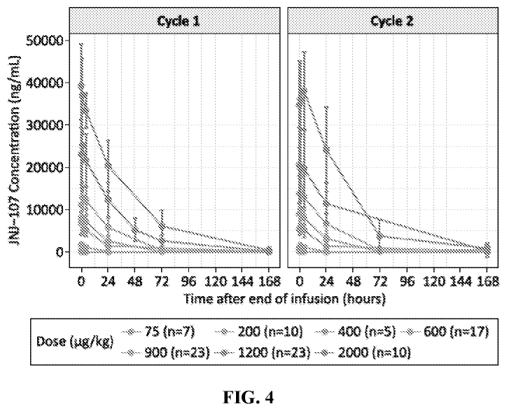

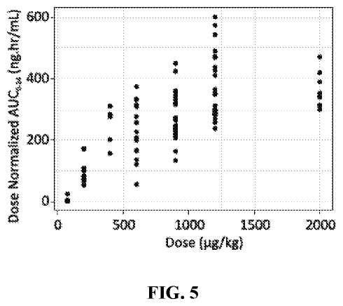

The preliminary PK of ANTIBODY A appeared to exhibit target-mediated drug disposition with rapid decline in serum concentrations (half-life: ˜13 h at 600 m/kg; ˜24 h at doses ≥1200 m/kg) (FIG. 4). No accumulation was observed after multiple biweekly dosing. Based on the limited data, target saturation was noted at around 1200 m/kg. Increases in mean Cₘₐₓ and AUC_{0-24 h} (FIG. 5) were more than dose-proportional at doses <1200 μm/kg and dose proportional at doses ≥1200 μm/kg. Immunogenicity data showed a low incidence of antibodies to ANTIBODY A (approximately 10.5%).

Pharmacodynamics Results

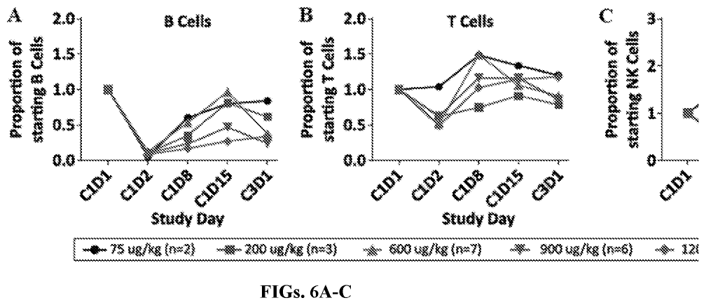

A dose-independent margination of B cells, T cells, and natural killer (NK) cells was observed following ANTIBODY A infusion, with dose-dependent B cell recovery consistent with observations for competitor anti-CD40 agonist antibodies. NK cells and T cells decreased in the peripheral blood following infusion at all doses tested, with the exception of the lowest dose (75 μm/kg); levels of both recovered fully by study day 8. See FIGS. 6A-C.

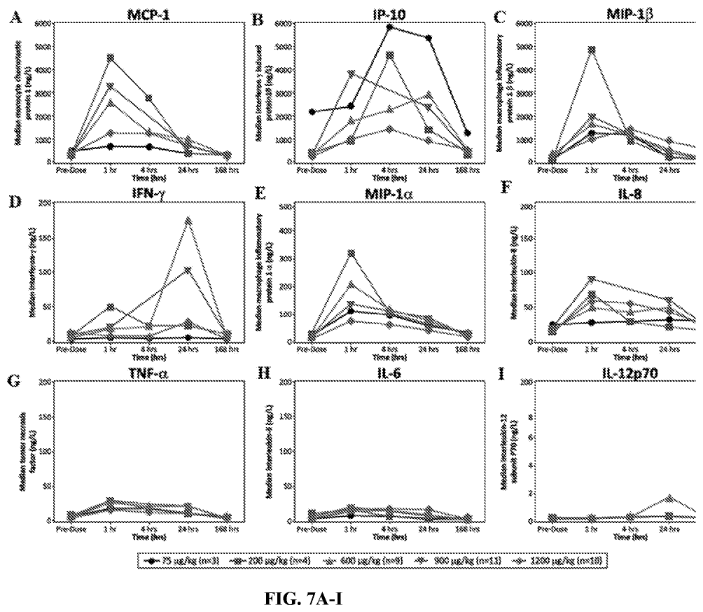

Peripheral chemokines (MCP-1, IP-10, and MIP-1β) were prominent in the peripheral blood, peaking 1-4 h post-infusion, which is consistent with myeloid cell activation. Cytokines (IFN-γ, TNF-α and IL12p70) and chemokines (MIP-la and IL-8) were also observed, but to a lesser extent. Levels of IL-6, which has been associated with the induction of cytokine storm with other CD40 agonist antibodies, were not abundant following infusion with ANTIBODY A. See FIGS. 7A-I.

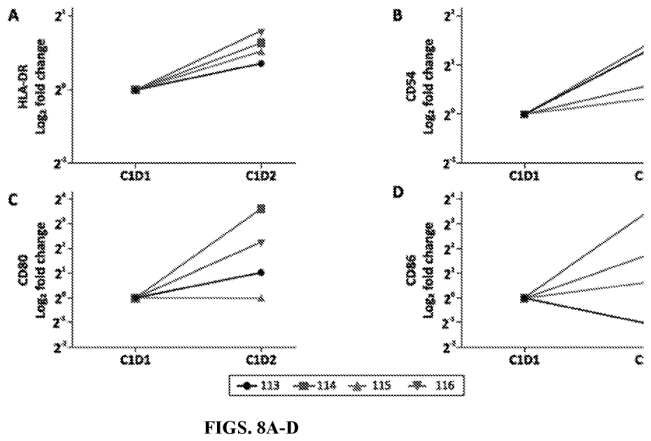

Phenotypic staining of peripheral B cells showed an increase in the fluorescence intensity of the activation/maturation markers (HLA-DR, CD54, CD80, and CD86), consistent with data from other agonist antibodies (FIGS. 8A-D).

Clinical Activity Results

Early evidence of clinical activity included a partial response in a patient with renal cell carcinoma and 10 patients with prolonged stable disease >6 months.

Discussion

The CD40 agonist ANTIBODY A has a manageable safety profile with favorable PK and PD properties. Preliminary ANTIBODY A PK is linear and dose proportional at doses ≥1200 μg/kg with moderate variability. ANTIBODY A led to increased levels of selected chemokines, notably MCP-1 and IP-10, and margination of B cells, T cells and NK cells post-infusion, with subsequent recovery. Remaining peripheral B cells exhibited increased activation/maturation markers.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present inventions as defined by the specific description.

1. A method of providing clinically proven safe administration of an anti-CD40 antibody to a human subject diagnosed with an advanced solid tumor, comprising intravenously administering to the subject a pharmaceutical composition comprising the antibody and a pharmaceutically acceptable carrier, wherein the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 1, 2, and 3, respectively, and the light chain variable region comprising light chain complementarity determining regions (LCDRs) LCDR1, LCDR2 and LCDR3 amino acid sequences of SEQ ID NOs: 4, 5, and 6, respectively, and wherein a total dosage of the antibody administered is 50 μg/kg to 2500 μg/kg, preferably 75 μg/kg to 2000 μg/kg, body weight of the subject per administration.

2. The method of claim 1, wherein the antibody comprises a heavy chain variable region (VH) having the amino acid sequence of SEQ ID NO: 7 and a light chain variable region (VL) having the amino acid sequences of SEQ ID NO: 8.

3. The method of claim 1, wherein the antibody comprises a heavy chain (HC) having the amino acid sequence of SEQ ID NO: 9 and a light chain (LC) having the amino acid sequences of SEQ ID NO: 10.

4. The method of claim 1, wherein the total dosage of the anti-CD40 antibody administered per administration is 75 μg/kg, 200 μg/kg, 400 μg/kg, 600 μg/kg, 700 μg/kg, 800 μg/kg, 900 μg/kg, 1000 μg/kg, 1100 μg/kg, 1200 μg/kg, 1300 μg/kg, 1400 μg/kg, 1500 μg/kg, 1800 μg/kg, or 2000 μg/kg body weight of the subject, or any dosage in between.

5. The method of claim 1, wherein the total dosage of the pharmaceutical composition is intravenously administered to the human subject over about 2 hours, preferably the pharmaceutical composition is intravenously administered to the human subject repeatedly, more preferably once every two weeks.

6. The method of claim 1, further comprising administering to the human subject a therapeutic agent before or after the administration of the anti-CD40 antibody, preferably the therapeutic agent is selected for the group consisting of corticosteroid, antihistamine, antipyretic, H₂-antagonist, and antiemetic.

7. The method of claim 1, further comprising administering to the subject an effective amount of at least one of Cetirizine and Montelukast in combination with the anti-CD40 antibody, to thereby reduce infusion-related reactions (IRRS) or reaction of pruritus, preferably cetirizine and montelukast are administered no earlier than 3 days before and no later than 3 days after the administration of the anti-CD40 antibody.

8. The method of claim 1, wherein the anti-CD40 antibody has an in vivo half-life of about 10-16 hours, preferably about 13 hours, when the total dosage of the anti-CD40 antibody administered per administration is 600 μg/kg body weight of the subject, or the anti-CD40 antibody has an in vivo half-life of about 20-28 hours, preferably 24 hours, when the total dosage of the anti-CD40 antibody administered per administration is no less than 1200 μg/kg body weight of the subject,.

9. The method of claim 1, wherein the human subject has no accumulation of the anti-CD40 antibody when the pharmaceutical composition is intravenously administered to the human subject repeatedly.

10. The method of claim 1, wherein the administration of the anti-CD40 results in target saturation when the total dosage of the anti-CD40 antibody administered per administration is about 1000-1400 μg/kg body weight of the subject, preferably 1200 μg/kg body weight of the subject.

11. The method of claim 1, wherein the administration of the anti-CD40 antibody causes, in the peripheral blood of the human subject, a margination of one or more cells selected from the group consisting of B cells, T cells, and natural killer (NK) cells, and a subsequent recovery thereof.

12. The method of claim 1, wherein the administration of the anti-CD40 antibody achieves, in the peripheral blood of the human subject, an increase of one or more chemokines selected from the group consisting of MCP-1, IP-10, MIP-1β, MIP-1α, and IL-8.

13. The method of claim 1, wherein the administration of the anti-CD40 antibody achieves, in the peripheral blood of the human subject, an increase of one or more cytokines selected from the group consisting of IFN-γ, TNF-α, and IL12p70.

14. The method of 1, wherein the administration of the anti-CD40 antibody results an increase of one or more activation markers on peripheral blood B lymphocytes, wherein the activation marker is selected from the group consisting of HLA-DR, CD54, CD80, and CD86.

15. The method of claim 1, wherein the human subject has at least a partial response or has a prolonged stable disease for 6 or more months.

16. A method of providing clinically proven safe administration of an anti-CD40 antibody to a human subject diagnosed with an advanced solid tumor, comprising intravenously administering to the subject a pharmaceutical composition comprising the antibody and a pharmaceutically acceptable carrier, wherein the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 1, 2, and 3, respectively, and the light chain variable region comprising light chain complementarity determining regions (LCDRs) LCDR1, LCDR2 and LCDR3 amino acid sequences of SEQ ID NOs: 4, 5, and 6, respectively, wherein a total dosage of the antibody administered is about 600 μg/kg to about 1200 μg/kg, such as 600 μg/kg, 700 μg/kg, 800 μg/kg, 900 μg/kg, 1000 μg/kg, 1100 μg/kg, 1200 μg/kg, body weight of the subject per administration, preferably the human subject is diagnosed with non-small cell lung cancer (NSCLC), pancreatic cancer, or cutaneous melanoma.

17. The method of claim 16, wherein the antibody comprises a heavy chain variable region (VH) having the amino acid sequence of SEQ ID NO: 7 and a light chain variable region (VL) having the amino acid sequences of SEQ ID NO: 8.

18. The method of claim 16, wherein the antibody comprises a heavy chain (HC) having the amino acid sequence of SEQ ID NO: 9 and a light chain (LC) having the amino acid sequences of SEQ ID NO: 10.
